(19) United States
(12) Patent Application Publication
(10) Pub. No.: US 2005/0266054 A1
(43) Pub. Date: Dec. 1, 2005

(54) Process of manufacturing antiseptic and scented clothe with cooling effect

(76) Inventor: Tsung-Kuei Tsai, Taoyuan City (TW)

Correspondence Address: Tsung-Kuei Tsai, P.O. Box 6-57, Junghe, Taipei 235, TW

(21) Appl. No.: 10/852,187

(22) Filed: May 25, 2004

(51) Int. Cl. A61K 9/70; A61K 9/14

(52) U.S. Cl. 424/443; 442/123

(57) ABSTRACT

The present invention is a process of manufacturing an antibacterial, scented clothe with a cooling effect, comprising a basic material united with an additive combined liquid formula. The composition of said combined solvent comprises of an incense treatment agent, a processed cooling disposal agent, a adhesion agent, water and an antiseptic deodorizing agent whereby applied to the basic material (fabric, flat-woven, needle-woven, non-woven fabrics etc.) by means of soaking, spraying or other suitable means, thereafter volatilizing the water content in the basic material, thus creating the antibacterial, scented clothe with a cooling effect.

### BACKGROUND OF THE INVENTION

(a) Field of Invention

The present invention relates to a process of manufacturing an antibacterial, scented clothe with a cooling effect, comprising of a basic material (fabric, flat-woven, needle-woven, non-woven fabrics etc.) united with an additive combined liquid formula. The composition of said combined solvent comprises of a antiseptic deodorizing agent, an incense treatment agent, a processed cooling disposal agent, a adhesion agent and water, whereby applied to the basic material (fabric, flat-woven, needle-woven, non-woven fabrics etc.) by means of soaking, spraying or other suitable means, thus creating the antibacterial, scented clothe with a cooling effect.

(b) Description of the Prior Art

With the improvement of technology and the satisfaction of materialistic living, people are slowly beginning to value quality of life, thus the development of one after another mood creating items, lighting products etc. including fabric.

Moreover, the conventional fabric on the market is unable to dissipate perfume, except continually spraying to maintain the scent, therefore unpopular among consumers.

Thus, the motive of the present invention as described hereunder, relates to a process of manufacturing an antibacterial, scented clothe with a cooling effect.

### SUMMARY OF INVENTION

A primary objective of the present invention is a process of manufacturing an antibacterial, scented clothe with a cooling effect.

A underlining clothe is treated (i.e. clothe hats, shoe padding, lavatory seat padding etc.), whereby includes vanilla (achieving scent dissipation), a antiseptic deodorizing agent (achieving anti-bacteria) and other compositions are included, thus achieving the effect of antibacterial, scented, cooling etc.

Clearer improvement and practicality, listed:
1. Improving users comfort.
2. Breaking a capsule by friction and releasing vanilla essence, thereby achieving long lasting scent effect and preventing rapid emanation.
3. Including anti-bacteria, thus high practicality
4. Comprises industrial competition.
5. Containing commercial value.
6. Displaying novelty.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a flow chart block diagram according to the present invention.
FIG. 2 shows a flow chart block diagram according to the present invention.
FIG. 3 shows a flow chart block diagram according to the present invention.

Appendix 1 shows a developmental testing report by the AEGIS Microbe Test Center in Asia according to the present invention.
Appendix 2 shows a composition ratio chart according to the present invention.
Appendix 3 shows a material safety data sheet by AEGIS Environmental according to the present invention.
Appendix 4 shows a certificate by the United States Environmental Protection Agency according to the present invention.
Appendix 5 shows a composition ratio chart according to the present invention.
Appendix 6 shows a material safety data sheet by Bayer according to the present invention.
Appendix 7 shows a graph according to the present invention.

### BREIF DESCRIPTION OF THE PREFERRED EMBODIMENT

To better understand the present invention, detailed descriptions shall be given with the accompany drawings hereunder.

As shown in FIG. 1 and enclosed appendix 1, 2, 3, 4, 6 and 7, whereby, a process of manufacturing an antibacterial and scented clothe with a cooling effect, comprising a basic material (underlining clothe 6, non-woven fabric etc.) united with an additive combined liquid formula. The composition of said combined solvent comprises of an incense treatment agent 1 (approximately 5%), a processed cooling disposal agent 2 (approximately 5%), a adhesion agent 3 (approximately 5%), water 4 (approximately 80%) and an antiseptic deodorizing agent 5 (approximately 5%), thus achieving antibacterial, scented and cooling effects. In addition to applying the underlining clothe 6 with the characteristics of the antiseptic deodorizing agent 5 (achieving antibacterial effects) and the incense treatment agent 1 (consisting suicide (silicone), further employing melamine (adhesion agent), allowing vanilla essence to be coated by the silicone to achieve a micro capsule form. Through friction by usage, the capsules break and release the vanilla essence, thereby achieving long lasting scented effect that prevents rapid emanation. Therefore, along with the processed cooling disposal agent 2 (applied to absorb heat to attain cooling effect), the aforesaid invention ultimately achieves antibacterial deodorizing, cooling and scented effects;

The flow diagram of the aforesaid invention comprise the following steps:
(a) Combining the incense treatment agent 1 (approximately 5%), a processed cooling disposal agent 2 (approximately 5%), a adhesion agent 3 (approximately 5%), water 4 (approximately 80%) and an antiseptic deodorizing agent 5 (approximately 5%), creating the combined solvent.
(b) Incorporating the combined solvent with the basic material (underlining clothe 6, non-woven fabric etc.) by applying soaking, spraying or other suitable means, thus achieving equal distribution.
(c) Manufacturing the antibacterial, scented clothe with cooling effect, thus completing a finished product 8.

As shown in FIG. 2, the above mentioned invention can further proceed with a aridity 7 process, whereby volatilizing the water content in the basic material, allowing the combined solvent to be more equally distributed on the basic material, thus improving product quality.

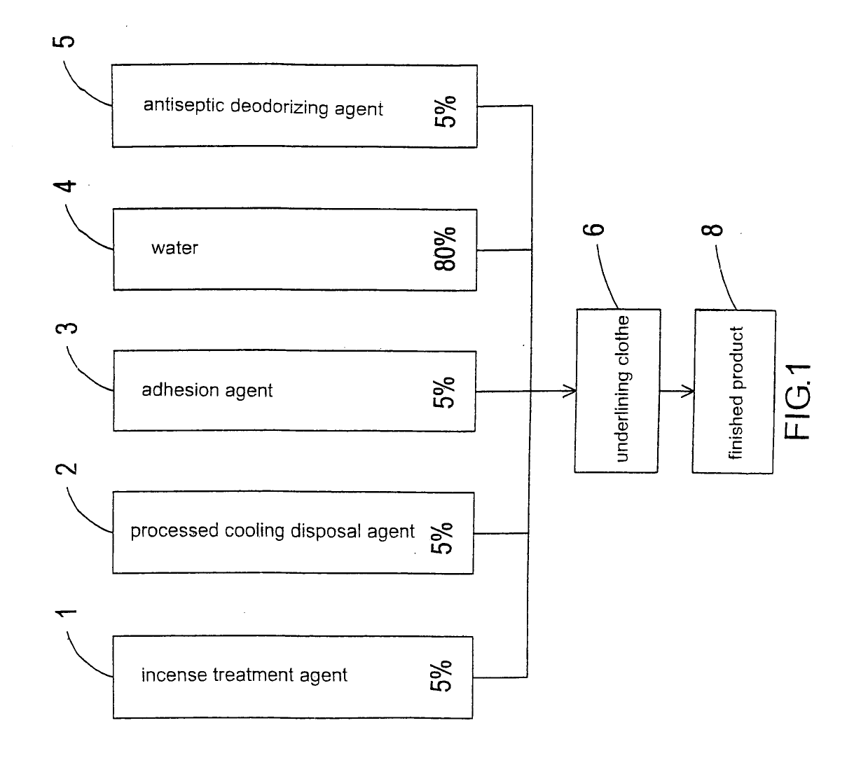

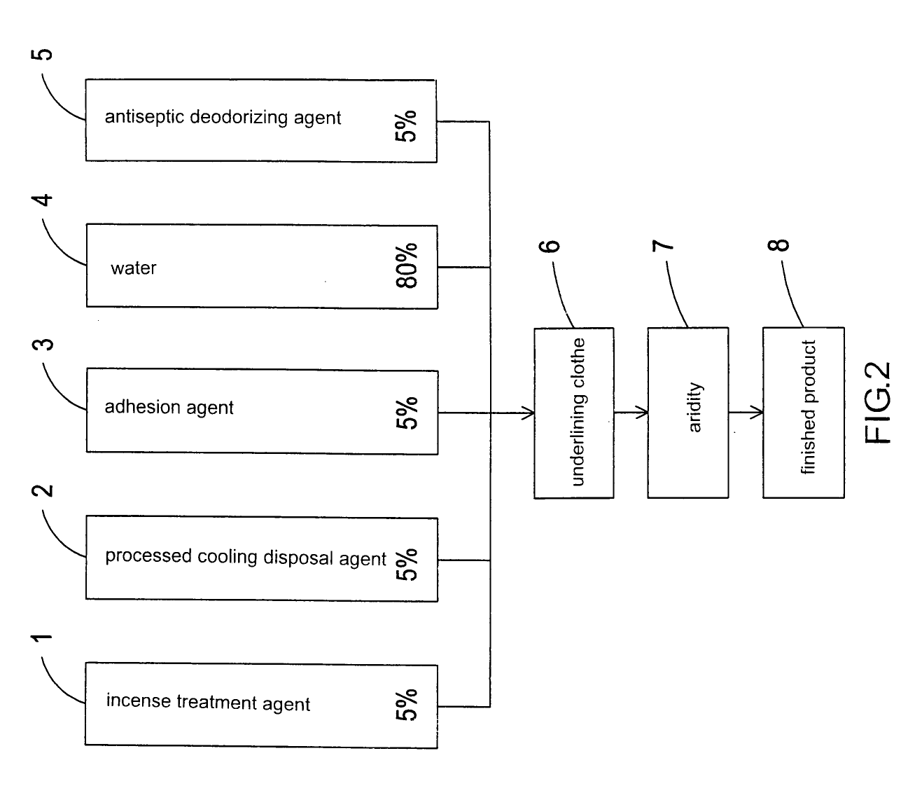

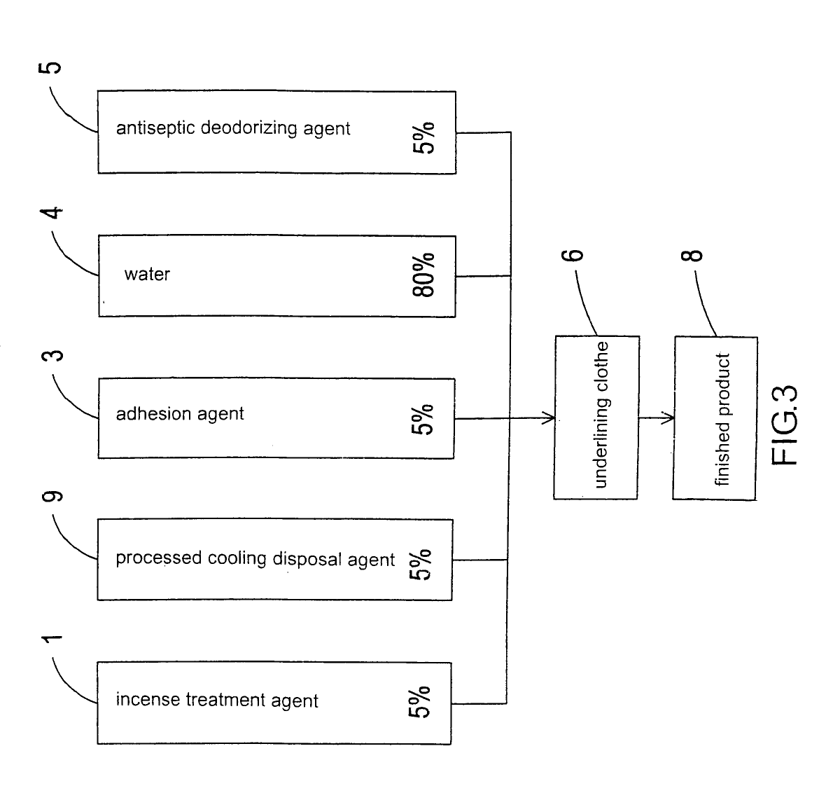

As shown in FIG. 3 and enclosed appendix 5, whereby further altering the processed cooling disposal agent 2 into a processed heating disposal agent 9 to retain heat, thus achieving a heat retaining function.

It is of course to be understood that the embodiments described herein is merely illustrative of the principles of the invention and that a wide variety of modifications thereto may be effected by persons skilled in the art without departing from the spirit and scope of the invention as set forth in the following claims.

1. A process of manufacturing an antibacterial, scented clothe with a cooling effect, having of a basic material (fabric, flat-woven, needle-woven, non-woven fabrics etc.) united with an additive combined liquid formula, comprising:
an incense treatment agent (approximately 5%), a processed cooling disposal agent (approximately 5%), a adhesion agent (approximately 5%), water (approximately 80%) and an antiseptic deodorizing agent (approximately 5%), thus achieving antibacterial, scented and cooling effects, moreover, applying the underlining clothe with the characteristics of the antiseptic deodorizing agent, thus achieving antibacterial effects, further, the incense treatment agent, allows vanilla essence to be coated by the silicone to achieve a micro capsule form, and through friction, the capsules break and release the vanilla essence thereafter, thus achieving long lasting scented effect that prevents rapid emanation along with the processed cooling disposal agent which is applied to absorb heat to attain cooling effect, therefore the above mentioned invention ultimately achieves antibacterial deodorizing, cooling and scented effects.

2. The process of manufacturing an antibacterial, scented clothe with a cooling effect as recited in claim 1, comprises the following steps;
(1) combining the incense treatment agent (approximately 5%), a processed cooling disposal agent (approximately 5%), a adhesion agent (approximately 5%), water (approximately 80%) and an antiseptic deodorizing agent (approximately 5%), creating the combined solvent;
(2) incorporating the combined solvent with the basic material (underlining clothe, non-woven fabric etc.) by applying soaking, spraying or other suitable means, thus achieving equal distribution.
(3) creating the antibacterial, scented clothe with cooling effect, thus completing a finished product.

3. The process of manufacturing an antibacterial, scented clothe with a cooling effect as recited in claim 1, whereby volatilizing the water content in the basic material, allowing the combined solvent to be more equally distributed on the basic material, thus improving product quality.

4. The process of manufacturing an antibacterial, scented clothe with a cooling effect as recited in claim 1, whereby further altering the processed cooling disposal agent into a processed heating disposal agent to retain heat, thus achieving a heat retaining function.
